(19) United States
(12) Patent Application Publication
(10) Pub. No.: US 2009/0312396 A1
(43) Pub. Date: Dec. 17, 2009

(54) METHODS FOR CANCER TREATMENT USING TAK1 INHIBITORS

(75) Inventors: Kate Byth, Waltham, MA (US); Sangeetha Palakurthi, Waltham, MA (US); Lihua Yu, Waltham, MA (US); Qi Zang, Waltham, MA (US)

Correspondence Address: ASTRAZENECA R&D BOSTON, 35 GATEHOUSE DRIVE, WALTHAM, MA, 02451-1215, US

(73) Assignee: AstraZeneca AB, 85 Sodertalje (SE)

(21) Appl. No.: 12/373,001

(22) PCT Filed: Jul. 10, 2006
(86) PCT No.: PCT/GB07/02568

§ 371 Date: Jan. 8, 2009

Related U.S. Application Data
(60) Provisional application No. 60/806,845, filed on Jul. 10, 2006.

(51) Int. Cl. A61K 31/7088; C12Q 1/68; A61K 31/496; A61K 31/5377; A61K 31/4535; A61K 31/381; A61P 35/00

(52) U.S. Cl. 514/44 A; 435/6; 514/252.13; 514/231.5; 514/326; 514/448

(57) ABSTRACT

The invention includes, in part, a method of inhibiting lymphoid tumour cell proliferation by contacting the lymphoid with a TAK1 inhibitor.

### FIELD OF THE INVENTION

The present invention relates to the treatment of cancer.

### BACKGROUND OF THE INVENTION

Lymphoid (B-cell and T-cell) tumours account for a significant proportion of human malignancies. The spectrum of different, but related, B cell malignancies includes B-cell acute lymphocytic leukemia (B-ALL), B-cell chronic lymphocytic leukemia (B-CLL), B-cell chronic myelogenous leukemia (B-CML), B-cell prolymphocytic leukemia (B-PLL), hairy cell leukemia (HCL), various B-cell non-Hodgkin's lymphomas (B-NHLs) (including diffuse large B cell lymphoma (DLBCL), Follicular Lymphoma (FCL or FL), mantle cell lymphoma (MCL), marginal zone lymphoma (MZL), Primary effusion lymphoma (PEL)) and Multiple Myeloma (MM). The spectrum of T-cell malignancies includes T-cell leukaemia, peripheral T-cell lymphoma (PTCL), T-cell lymphoblastic lymphoma (T-CLL), cutaneous T-cell lymphoma (CTCL) and adult T-cell lymphoma (ATCL). Treatment of non-Hodgkin's lymphomas including both B-cell and T-cell tumours, chronic lymphocytic leukemias (CLL) and multiple myelomas (MM) is frequently unsatisfactory and attempts to link clinical or cellular characteristics of the disease to prognosis and treatment have met with difficulties.

### SUMMARY OF THE INVENTION

The present invention is based, in part, on methods that can be used to treat a patient having cancer with a TGF-beta activated kinase 1 (TAK1, MAP3K7) inhibitor. The invention further includes selecting patients having cancer who would be responsive to treatment with a TAK1 inhibitor. Moreover, the invention includes methods for determining for the presence of one or more deregulated TAK1 signal transduction molecules in a tumour cell. The presence of a deregulated TAK1 signal transduction molecule indicates that a TAK1 inhibitor should be administered.

In one aspect, the invention includes inhibiting B cell tumour cell proliferation by contacting a B cell tumour cell with a TAK1 inhibitor. The B cell tumour can be a non-Hodgkin's lymphoma, a chronic lymphocytic leukaemia, or a multiple myeloma.

In another aspect, the invention includes inhibiting the growth of a solid tumour by contacting the tumour with a TAK1 inhibitor. The solid tumour can be a tumour of the head and neck, breast, ovary, lung, pancreas, colon, prostate, liver, kidney or skin.

In another aspect, the invention includes inhibiting proliferation of a T-cell leukemia and T-cell lymphoma by contacting the T-cell leukaemia and T-cell lymphoma with a TAK1 inhibitor. A T cell leukemia can include T-cell acute lymphoblastic leukemia (T-ALL), T-lymphoblastic lymphoma, T-CLL, CTCL or other T-NHLs. The TAK1 inhibitor can be administered either as a single agent or in combination with other anti-cancer agents or anti-cancer antibodies.

In another aspect, the invention includes a method of treating cancer. In one aspect, the invention includes a method of treating a patient having a B cell tumour by administering a TAK1 inhibitor. The B-cell tumour can be a non-Hodgkin's lymphoma, a chronic lymphocytic leukaemia or a multiple myeloma. In one example, the non-Hodgkin's lymphoma can be a follicular lymphoma, a diffuse large B cell lymphoma (DLBCL) of activated B cell (ABC) type, a diffuse large B cell lymphoma (DLBCL) of germinal center B cell (GCB) type, a mantle zone lymphoma (MZL), Mantle cell lymphoma (MCL), or MALT Lymphoma.

In another example, the non-Hodgkin's lymphoma has a t(14;18)(q32;q21) translocation, t(11;18)(q21;q21) translocation, t(1;14)(p22;q32), amplification of chromosome 18, addition of chromosome 18q21, amplification of chromosome 6, or amplification, as defined by comparative genomic hybridisation, of specific regions including BCL-10, CARD11, TRAF6 and TAK1.

In another aspect, the invention includes treating a patient having a solid tumour by administering a TAK1 inhibitor. The solid tumour can be a tumour of the head and neck, breast, ovary, lung, pancreas, colon, prostate, or skin.

In another aspect, the invention includes treating a patient having a T-cell leukemia by contacting the T-cell leukemia with a TAK1 inhibitor. A T-cell leukemia can include T-cell acute lymphoblastic leukemia (T-ALL), T-lymphoblastic lymphoma, T-CLL, CTCL or other T-NHLs.

In yet another aspect, the invention includes a method of treating a patient having a deregulated TAK1 signalling transduction molecule by administering a TAK1 inhibitor. The TAK1 signalling transduction molecule can be MALT1, BCL-10, TAB1, TAB2, TRAF6, TRAF2, TAK1, CARD11, IRAK1, IRAK4, API1, API2, API3, API4 (survivin), BCL2 or NFkB target genes. The TAK1 signalling molecule can be a DNA molecule in either mutated or amplified or translocated form. The TAK1 signaling molecule can be a protein in its naïve form or modified, either by phosphorylation, ubiquitination, changed in sequence due to mutation, etc. The TAK1 signaling molecule can also be monitored by its subcellular localization. One example of such alterations in subcellular localization is shown by increased nuclear localization of BCL10 due to the gene amplification in a diffuse large B cell lymphoma with IGH-BCL2 fusion (Ye H et. al Haematologica. 2006; 91 (6 Suppl)).

In another embodiment, a deregulated TAK1 signalling transduction molecule can be one or more of the molecules listed in Table 1 or Table 2 below.

TABLE 1

| Gene | | | |
|---|---|---|---|
| Symbol | Entrez | Pathway | U133A/B |
| RANK | 8792 | Rank-L | 207037_at; 238846_at; |
| RANKL | 8600 | Rank-L | 210643_at; 211153_s_at; 241248_at; |
| TNFR1 | 7132 | TNF | 207643_s_at; 241944_x_at; |
| TNFR2 | 7133 | TNF | 203508_at; |
| TRADD | 8717 | TNF | 1729_at; 205641_s_at; 213443_at; |
| TANK | 10010 | TNF | 207616_s_at; 209451_at; 210458_s_at; 241191_at; 243376_at; |
| GCK | 2645 | TNF | 211167_s_at; |
| RIP | 3267 | TNF | 213926_s_at; 213928_s_at; |
| RAIDD | 8738 | TNF | 209833_at; 242638_at; |
| PROCASP2 | 835 | TNF | 208050_s_at; 209811_at; 209812_x_at; 211140_s_at; 34449_at; |
| | | | 226032_at; 226036_x_at; |
| UBC13 | 7334 | TNF | 201523_x_at; 201524_x_at; 212751_at; |
| UEV1A | 7335 | TNF | 240129_at; |
| TLR2 | 7097 | TLRs | 204924_at; |
| TLR4 | 7099 | TLRs | 221060_s_at; 224341_x_at; 232068_s_at; 240948_at; |
| TLR3 | 7098 | TLRs | 206271_at; |
| TLR7 | 51284 | TLRs | 220146_at; 222952_s_at; |
| TLR9 | 54106 | TLRs | 223903_at; |
| TLR10 | 81793 | TLRs | 223750_s_at; 223751_x_at; |
| MYD88 | 4615 | TLRs | 209124_at; |
| TOLLIP | 54472 | TLRs | 217930_s_at; 222469_s_at; 233881_s_at; |
| IRAK | 3654 | TLRs | 201587_s_at; |
| TIRAP | 114609 | TLRs | 236687_at; 239796_x_at; |
| PKR | 5610 | TLRs | 204211_x_at; |
| TBK1 | 29110 | TLRs | 218520_at; |
| TGFB | 7040 | TGF-beta | 203084_at; 203085_s_at; |
| IL1R | 3554 | IL1 | 202948_at; 215561_s_at; |
| TOLLIP | 54472 | IL1 | 217930_s_at; 222469_s_at; 233881_s_at; |
| MYD88 | 4615 | IL1 | 209124_at; |
| CD19 | 930 | B-Cell | 206398_s_at; |
| CD22 | 933 | B-Cell | |
| CD45 | 5788 | B-Cell | 207238_s_at; 212587_s_at; 212588_at; |
| CD79A | 973 | B-Cell | 205049_s_at; |
| CD79B | 974 | B-Cell | 205297_s_at; |
| LYN | 4067 | B-Cell | 202625_at; 202626_s_at; 210754_s_at; 239555_at; 243633_at; |
| SHP1 | 8431 | B-Cell | 206410_at; |
| SYK | 6850 | B-Cell | 207540_s_at; 209269_s_at; 226068_at; 244023_at; |
| GAB1 | 2549 | B-Cell | 207112_s_at; 225998_at; 242572_at; |
| GAB2 | 9846 | B-Cell | 203853_s_at; 238403_at; 238405_at; 241004_at; |
| SHP2 | 5781 | B-Cell | 205867_at; 205868_s_at; 209895_at; 209896_s_at; 212610_at; |
| CSK | 1445 | B-Cell | 202329_at; |
| PAG | 55824 | B-Cell | 225622_at; 225626_at; |
| VAV | 7409 | B-Cell | 206219_s_at; |
| VAV2 | 7410 | B-Cell | 205536_at; 205537_s_at; 226063_at; |
| GRB2 | 2885 | B-Cell | 215075_s_at; 223049_at; 228572_at; |
| BLNK | 29760 | B-Cell | 207655_s_at; 244172_at; |
| SOS | 6654 | B-Cell | 212777_at; 212780_at; 229261_at; 230337_at; 232883_at; 242018_at; |
| | | | 242682_at; |
| PLCG2 | 5336 | B-Cell | 204613_at; |
| PKCB | 5579 | B-Cell | 207957_s_at; 209685_s_at; 227817_at; 227824_at; 228795_at; 230437_s_at; |
| PRKCQ | 5588 | B-Cell | 210038_at; 210039_s_at; |
| PRKCQ | 5588 | T-Cell | 210038_at; 210039_s_at; |
| PKCB | 5579 | T-Cell | 207957_s_at; 209685_s_at; 227817_at; 227824_at; 228795_at; 230437_s_at; |
| CD28 | 940 | T-Cell | 206545_at; 211856_x_at; 211861_x_at; |
| CD3 | 917 | T-Cell | 206804_at; |
| CD3E | 916 | T-Cell | 205456_at; |
| CD3D | 915 | T-Cell | 213539_at; |
| CD4 | 920 | T-Cell | 203547_at; 216424_at; |
| UBC4 | 7322 | T-Cell | 201343_at; 201344_at; 201345_s_at; 215604_x_at; 240139_at; |
| UBC5 | 6923 | T-Cell | 200085_s_at; 213877_x_at; 200085_s_at; |
| CIAP1 | 329 | T-Cell | 202076_at; |
| CIAP2 | 330 | T-Cell | 210538_s_at; 230499_at; |
| CD8 | 925 | T-Cell | 205758_at; |
| CD8B | 926 | T-Cell | 207979_s_at; 215332_s_at; 230037_at; |
| CD45 | 5788 | T-Cell | 207238_s_at; 212587_s_at; 212588_at; |
| ZAP70 | 7535 | T-Cell | 214032_at; |
| FYN | 2534 | T-Cell | 210105_s_at; 212486_s_at; 216033_s_at; 217697_at; 243006_at; |
| CLTA4 | 1493 | T-Cell | 221331_x_at; 231794_at; 234362_s_at; 234895_at; 236341_at; |
| PI3k | 5286 | T-Cell | 213070_at; 226094_at; 235792_x_at; 241905_at; |
| PIK3C2B | 5287 | T-Cell | 204484_at; |
| PIK3C2G | 5288 | T-Cell | 215129_at; |
| PIK3C3 | 5289 | T-Cell | 204297_at; 215394_at; 232086_at; 239300_at; |
| PIK3CA | 5290 | T-Cell | 204369_at; 215212_at; |
| PIK3CB | 5291 | T-Cell | 212688_at; 217620_s_at; |
| PIM1 | 5292 | T-Cell | 209193_at; |
| PIK3CD | 5293 | T-Cell | 203879_at; 211230_s_at; |
| PIK3CG | 5294 | T-Cell | 206369_s_at; 206370_at; |
| VAV | 7409 | T-Cell | 206219_s_at; |
| VAV2 | 7410 | T-Cell | 205536_at; 205537_s_at; 226063_at; |
| VAV3 | 10451 | T-Cell | 218806_s_at; 218807_at; 224221_s_at; |
| ITK | 3702 | T-Cell | 211339_s_at; |
| SLP76 | 3937 | T-Cell | 205269_at; 205270_s_at; 244251_at; 244556_at; 244578_at; |
| LAT | 27040 | T-Cell | 209881_s_at; 211005_at; |
| PAG | 55824 | T-Cell | 225622_at; 225626_at; |
| CBL | 867 | T-Cell | 206607_at; 225231_at; 225234_at; 229010_at; 243475_at; |
| LCK | 3932 | T-Cell | 204890_s_at; 204891_s_at; |
| SHB | 6461 | T-Cell | 204656_at; 204657_s_at; 230459_s_at; 234794_at; 234795_at; 243595_at; |
| PLCG1 | 5335 | T-Cell | |
| LTBR | 4055 | LTBR | 203005_at; 232819_s_at; 243400_x_at; |
| NIK | 9020 | LTBR | 205192_at; |
| NIKBR | 83696 | LTBR | 221672_s_at; 221836_s_at; 56829_at; 229108_at; 237001_at; 237851_at; |
| GADD45A | 1647 | Suvival | 203725_at; |
| GADD45B | 4616 | Suvival | 207574_s_at; 209304_x_at; 209305_s_at; 213560_at; |
| GADD45G | 10912 | Suvival | 204121_at; |
| XIAP | 331 | Suvival | 206536_s_at; 225858_s_at; 225859_at; 228363_at; 235222_x_at; 243026_x_at; |
| BCL-XL | 598 | Suvival | 206665_s_at; 212312_at; 215037_s_at; 231228_at; |
| FLIP | 8837 | Suvival | 208485_x_at; 209508_x_at; 209939_x_at; 210563_x_at; 210564_x_at; 211316_x_at; |
| | | | 211317_s_at; 211862_x_at; 214486_x_at; 214618_at; 217654_at; 237367_x_at; |
| | | | 239629_at; |
| IAP | 330 | Suvival | 210538_s_at; 230499_at; |
| SURVIVIN | 332 | Suvival | 202094_at; 202095_s_at; 210334_x_at; |
| TNFRSF10C - DCR1 | 8794 | Death | 206222_at; 211163_s_at; |
| TRAF1 | 7185 | Death | 205599_at; 235116_at; |
| RELA | 5970 | Death | 201783_s_at; 209878_s_at; 230202_at; |
| TNFRSF1A - TNFR1 | 7132 | Death | 207643_s_at; 241944_x_at; |
| FADD | 8772 | Death | 202535_at; |
| CASP8 | 841 | Death | 207686_s_at; 213373_s_at; 231218_at; |
| CASP3 | 836 | Death | 202763_at; 236729_at; |
| BCL2 | 596 | Death | 203684_s_at; 203685_at; 207004_at; 207005_s_at; 232210_at; 232614_at; |
| | | | 237837_at; 244035_at; |
| CARMA1 | 84433 | Traf 2/6 | 223514_at; |
| | | Complex | |
| BCL10 | 8915 | Traf 2/6 | 205263_at; |
| | | Complex | |
| MALT1 | 10892 | Traf 2/6 | 208309_s_at; 210017_at; 210018_x_at; 238157_at; |
| | | Complex | |
| TRAF6 | 7186 | Traf 2/6 | 204413_at; |
| | | Complex | |
| TRAF2 | 7189 | Traf 2/6 | 205558_at; |
| | | Complex | |
| PAR1 | 2149 | PAR | 203989_x_at; |
| PAR4 | 5074 | PAR | 204004_at; 204005_s_at; 214090_at; 214237_x_at; 226223_at; 226231_at; |
| | | | 229515_at; |
| CRK | 1398 | SAPK/JNK | 202224_at; 202226_s_at; |
| CRKL | 1399 | SAPK/JNK | 206184_at; 212180_at; |
| SHC | 6464 | SAPK/JNK | 201469_s_at; 214853_s_at; |
| SHC2 | 53358 | SAPK/JNK | 206330_s_at; |
| SHC3 | 25759 | SAPK/JNK | 213464_at; |
| GRB2 | 2885 | SAPK/JNK | 215075_s_at; 223049_at; 228572_at; |
| SOS | 6654 | SAPK/JNK | 212777_at; 212780_at; 229261_at; 230337_at; 232883_at; 242018_at; |
| | | | 242682_at; |
| HPK2 | 9448 | SAPK/JNK | 206571_s_at; 218181_s_at; 222547_at; 222548_s_at; 238769_at; 244846_at; |
| TAB1 | 10454 | TAK1 | 203901_at; 233679_at; 235480_at; 235827_at; |
| | | Complex | |
| TAB2 | 23118 | TAK1 | 210284_s_at; 212184_s_at; 233957_at; 243557_at; |
| | | Complex | |
| TAB3 | 257397 | TAK1 | 227357_at; |
| | | Complex | |
| TAK1 | 6885 | TAK1 | 206853_s_at; 206854_s_at; 211536_x_at; 211537_x_at; |
| | | Complex | |
| NLK | 51701 | NFkB | 218318_s_at; 222589_at; 222590_s_at; |
| IKK1 | 1147 | NFkB | 209666_s_at; |
| IKK2 | 3551 | NFkB | 209341_s_at; 209342_s_at; 211027_s_at; |
| IKK3 | 8517 | NFkB | 209929_s_at; 36004_at; |
| NFKB | 4790 | NFkB | 209239_at; 239876_at; |
| RELA | 5970 | NFkB | 201783_s_at; 209878_s_at; 230202_at; |
| MKK4 | 6416 | JNK | 203265_s_at; 203266_s_at; |
| MKK7 | 5609 | JNK | 209951_s_at; 209952_s_at; 216206_x_at; 226023_at; 226053_at; |
| JNK | 5599 | JNK | 210477_x_at; 210671_x_at; 226046_at; 226048_at; 243280_at; |
| MAPK9 | 5601 | JNK | 203218_at; 210570_x_at; 225781_at; |
| MAPK10 | 5602 | JNK | 204813_at; |
| DUSP8 | 1850 | JNK | 206374_at; |
| IRS1 | 3667 | JNK | 204686_at; 235392_at; |
| SMC | 9918 | JNK | 201774_s_at; |
| HNRPK | 3190 | JNK | 200097_s_at; 200775_s_at; 200097_s_at; |
| TP53 | 7157 | JNK | 201746_at; 211300_s_at; 224185_at; |
| ATF2 | 1386 | JNK | 205446_s_at; 212984_at; |
| ELK1 | 2002 | JNK | 203617_x_at; 210376_x_at; 210850_s_at; |
| CJUN | 3725 | JNK | 201464_x_at; 201465_s_at; 201466_s_at; 213281_at; |
| NFAT4 | 4775 | JNK | 207416_s_at; 210555_s_at; 210556_at; 229223_at; |
| NFATC1 | 4772 | JNK | 208196_x_at; 209664_x_at; 210162_s_at; 211105_s_at; |
| NLK | 51701 | WNT | 218318_s_at; 222589_at; 222590_s_at; |
| CTBP1 | 1487 | WNT | 203392_s_at; 212863_x_at; 213980_s_at; 243180_at; |
| CBP | 1387 | WNT | 202160_at; 211808_s_at; 235858_at; 237239_at; |
| TCF | 3172 | WNT | 208429_x_at; 214832_at; 214851_at; 216889_s_at; 230914_at; |
| GROUCHO | 7091 | WNT | 204872_at; 214688_at; 216997_x_at; 233575_s_at; 235765_at; |
| HIPK2 | 28996 | WNT | 213763_at; 219028_at; 224016_at; 224065_at; 224066_s_at; 225097_at; |
| | | | 225115_at; 225116_at; 225368_at; 240294_at; |
| cMYB | 4602 | WNT | 204798_at; 215152_at; |
| ATF2 | 1386 | ALK | 205446_s_at; 212984_at; |
| CSX | 1482 | ALK | 206578_at; |
| GATA4 | 2626 | ALK | 205517_at; 230855_at; 243692_at; |
| MAP2K6 | 5608 | p38 | 205698_s_at; |
| p38MAPK | 5594 | p38 | 208351_s_at; 212271_at; 224620_at; 224621_at; 229847_at; 242106_at; |
| MNK1 | 8569 | p38 | 209467_s_at; 243256_at; |
| PRAK | 8550 | p38 | 212871_at; |
| HSP27 | 6294 | p38 | 201747_s_at; 201748_s_at; 213635_s_at; |
| MAPKAP2 | 9261 | p38 | 201460_at; 201461_s_at; 215050_x_at; |
| PLA2 | 5320 | p38 | 203649_s_at; |
| STAT1 | 6772 | p38 | 200887_s_at; 209969_s_at; AFFX-HUMISGF3A/M97935_3_at; AFFX- |
| | | | HUMISGF3A/M97935_5_at; AFFX-HUMISGF3A/M97935_MA_at; AFFX- |
| | | | HUMISGF3A/M97935_MB_at; 232375_at; AFFX-HUMISGF3A/M97935_3_at; AFFX- |
| | | | HUMISGF3A/M97935_5_at; AFFX-HUMISGF3A/M97935_MA_at; AFFX- |
| | | | HUMISGF3A/M97935_MB_at; |
| MAX | 4149 | p38 | 208403_x_at; 209331_s_at; 209332_s_at; 210734_x_at; 214108_at; 222174_at; |
| MYC | 4609 | p38 | 202431_s_at; 239931_at; 244089_at; |
| ELK1 | 2002 | p38 | 203617_x_at; 210376_x_at; 210850_s_at; |
| CHOP | 2521 | p38 | 200959_at; 215744_at; 217370_x_at; 231108_at; |
| MEF2 | 4205 | p38 | 208328_s_at; 212535_at; 214684_at; 239571_at; 242176_at; |
| MEF3 | 4206 | p38 | |
| MEF4 | 4207 | p38 | 205124_at; 209926_at; |
| MEF5 | 4208 | p38 | 207968_s_at; 209199_s_at; 209200_at; 236395_at; 239938_x_at; 239966_at; |
| | | | 244230_at; |
| MEF6 | 4209 | p38 | 203003_at; 203004_s_at; 225641_at; |
| ATF2 | 1386 | p38 | 205446_s_at; 212984_at; |
| MSK1 | 9252 | p38 | 204633_s_at; 204635_at; |
| HMG14 | 3150 | p38 | 200943_at; 200944_s_at; |
| CREB | 1385 | p38 | 204312_x_at; 204313_s_at; 204314_s_at; 214513_s_at; 243625_at; |

TABLE 2
TAK inhibitor sensitivity determining signature including the informative genes that differentiate 3 subclasses of DLBCL patients

| Gene | | | | Gene |
|---|---|---|---|---|
| Symbol | Entrez | Pathway | U133A/B | Group |
| TNFR1 | 7132 | TNF | 207643_s_at; | A |
| TNFR2 | 7133 | TNF | 203508_at; | A |
| TRADD | 8717 | TNF | 1729_at; 205641_s_at; | AA |
| TANK | 10010 | TNF | 207616_s_at; 209451_at; 210458_s_at; | EEE |
| RAIDD | 8738 | TNF | 209833_at; | B |
| PROCASP2 | 835 | TNF | 208050_s_at; 209811_at; 211140_s_at; 34449_at; 226032_at; | BBBBC |
| UBC13 | 7334 | TNF | 201523_x_at; 201524_x_at; 212751_at; | EEE |
| TLR2 | 7097 | TLRs | 204924_at; | E |
| TLR4 | 7099 | TLRs | 221060_s_at; 224341_x_at; 232068_s_at; | EEE |
| TLR7 | 51284 | TLRs | 220146_at; 222952_s_at; | EE |
| TLR10 | 81793 | TLRs | 223750_s_at; 223751_x_at; | FF |
| MYD88 | 4615 | TLRs | 209124_at; | C |
| TOLLIP | 54472 | TLRs | 217930_s_at; 233881_s_at; | ED |
| IRAK | 3654 | TLRs | 201587_s_at; | E |
| PKR | 5610 | TLRs | 204211_x_at; | E |
| TBK1 | 29110 | TLRs | 218520_at; | E |
| TGFB | 7040 | TGF-beta | 203085_s_at; | C |
| IL1R | 3554 | IL1 | 202948_at; | E |
| TOLLIP | 54472 | IL1 | 217930_s_at; 233881_s_at; | ED |
| MYD88 | 4615 | IL1 | 209124_at; | C |
| CD19 | 930 | B-Cell | 206398_s_at; | F |
| CD45 | 5788 | B-Cell | 207238_s_at; 212587_s_at; 212588_at; | AAA |
| CD79A | 973 | B-Cell | 205049_s_at; | F |
| CD79B | 974 | B-Cell | 205297_s_at; | F |
| LYN | 4067 | B-Cell | 202625_at; 202626_s_at; 210754_s_at; 239555_at; | EEED |
| SHP1 | 8431 | B-Cell | 206410_at; | B |
| SYK | 6850 | B-Cell | 207540_s_at; 209269_s_at; 226068_at; 244023_at; | FFFF |
| GAB1 | 2549 | B-Cell | 207112_s_at; 242572_at; | BE |
| SHP2 | 5781 | B-Cell | 205868_s_at; 209895_at; 209896_s_at; 212610_at; | DDEE |
| CSK | 1445 | B-Cell | 202329_at; | C |
| PAG | 55824 | B-Cell | 225622_at; 225626_at; | DD |
| VAV | 7409 | B-Cell | 206219_s_at; | C |
| VAV2 | 7410 | B-Cell | 205536_at; 226063_at; | FF |
| GRB2 | 2885 | B-Cell | 215075_s_at; 223049_at; | EE |
| BLNK | 29760 | B-Cell | 207655_s_at; 244172_at; | FF |
| SOS | 6654 | B-Cell | 212777_at; 212780_at; 229261_at; 230337_at; | CCDDDE |
| | | | 232883_at; 242018_at; | |
| PLCG2 | 5336 | B-Cell | 204613_at; | C |
| PKCB | 5579 | B-Cell | 207957_s_at; 209685_s_at; 227817_at; 227824_at; 228795_at; | DDDDD |
| PRKCQ | 5588 | B-Cell | 210038_at; 210039_s_at; | AB |
| PRKCQ | 5588 | T-Cell | 210038_at; 210039_s_at; | AB |
| PKCB | 5579 | T-Cell | 207957_s_at; 209685_s_at; 227817_at; 227824_at; 228795_at; | DDDDD |
| CD28 | 940 | T-Cell | 206545_at; 211856_x_at; 211861_x_at; | AAA |
| CD3 | 917 | T-Cell | 206804_at; | A |
| CD3E | 916 | T-Cell | 205456_at; | A |
| CD3D | 915 | T-Cell | 213539_at; | A |
| CD4 | 920 | T-Cell | 203547_at; | A |
| UBC4 | 7322 | T-Cell | 201343_at; 201345_s_at; 215604_x_at; 240139_at; | FFDC |
| UBC5 | 6923 | T-Cell | 200085_s_at; 200085_s_at; | EE |
| CIAP1 | 329 | T-Cell | 202076_at; | E |
| CIAP2 | 330 | T-Cell | 210538_s_at; 230499_at; | EE |
| CD8 | 925 | T-Cell | 205758_at; | A |
| CD8B | 926 | T-Cell | 207979_s_at; 215332_s_at; | AE |
| CD45 | 5788 | T-Cell | 207238_s_at; 212587_s_at; 212588_at; | AAA |
| ZAP70 | 7535 | T-Cell | 214032_at; | A |
| FYN | 2534 | T-Cell | 210105_s_at; 212486_s_at; 216033_s_at; 217697_at; 243006_at; | AAABA |
| CLTA4 | 1493 | T-Cell | 221331_x_at; 231794_at; 234362_s_at; 236341_at; | AAAA |
| PI3k | 5286 | T-Cell | 213070_at; 235792_x_at; 241905_at; | EDD |
| PIK3C2B | 5287 | T-Cell | 204484_at; | D |
| PIK3C2G | 5288 | T-Cell | 215129_at; | B |
| PIK3C3 | 5289 | T-Cell | 204297_at; 215394_at; 239300_at; | EBA |
| PIK3CA | 5290 | T-Cell | 204369_at; 215212_at; | EE |
| PIK3CB | 5291 | T-Cell | 212688_at; 217620_s_at; | DD |
| PIM1 | 5292 | T-Cell | 209193_at; | E |
| PIK3CD | 5293 | T-Cell | 203879_at; 211230_s_at; | CC |
| PIK3CG | 5294 | T-Cell | 206369_s_at; 206370_at; | DD |
| VAV | 7409 | T-Cell | 206219_s_at; | C |
| VAV2 | 7410 | T-Cell | 205536_at; 226063_at; | FF |
| VAV3 | 10451 | T-Cell | 218806_s_at; 218807_at; | EE |
| ITK | 3702 | T-Cell | 211339_s_at; | A |
| SLP76 | 3937 | T-Cell | 205269_at; 205270_s_at; 244251_at; 244556_at; | AAAA |
| LAT | 27040 | T-Cell | 209881_s_at; 211005_at; | AA |
| PAG | 55824 | T-Cell | 225622_at; 225626_at; | DD |
| CBL | 867 | T-Cell | 206607_at; 225231_at; 225234_at; 229010_at; 243475_at; | BCCCD |
| LCK | 3932 | T-Cell | 204890_s_at; 204891_s_at; | AA |
| SHB | 6461 | T-Cell | 204656_at; 204657_s_at; | DB |
| LTBR | 4055 | LTBR | 203005_at; | B |
| NIK | 9020 | LTBR | 205192_at; | D |
| NIKBR | 83696 | LTBR | 221672_s_at; 221836_s_at; 56829_at; | CCC |
| GADD45A | 1647 | Suvival | 203725_at; | E |
| GADD45B | 4616 | Suvival | 207574_s_at; 209304_x_at; 209305_s_at; | EEE |
| XIAP | 331 | Suvival | 206536_s_at; 225858_s_at; 225859_at; 228363_at; | BEEEEE |
| | | | 235222_x_at; 243026_x_at; | |
| BCL-XL | 598 | Suvival | 206665_s_at; 212312_at; 215037_s_at; | EEE |
| FLIP | 8837 | Suvival | 208485_x_at; 209508_x_at; 209939_x_at; | AAAAAAAAA |
| | | | 210563_x_at; 210564_x_at; 211316_x_at; | BBAA |
| | | | 211317_s_at; 211862_x_at; 214486_x_at; | |
| | | | 214618_at; 217654_at; 237367_x_at; 239629_at; | |
| IAP | 330 | Suvival | 210538_s_at; 230499_at; | EE |
| SURVIVIN | 332 | Suvival | 202094_at; 202095_s_at; 210334_x_at; | EEB |
| TNFRSF10C - DCR1 | 8794 | Death | 206222_at; | B |
| TRAF1 | 7185 | Death | 205599_at; 235116_at; | DD |
| RELA | 5970 | Death | 201783_s_at; 209878_s_at; | CC |
| TNFRSF1A - TNFR1 | 7132 | Death | 207643_s_at; | A |
| FADD | 8772 | Death | 202535_at; | E |
| CASP8 | 841 | Death | 207686_s_at; 213373_s_at; | EA |
| CASP3 | 836 | Death | 202763_at; | E |
| BCL2 | 596 | Death | 203685_at; 207005_s_at; 232210_at; 232614_at; 244035_at; | EEEEE |
| CARMA1 | 84433 | Traf 2/6 | 223514_at; | E |
| | | Complex | | |
| BCL10 | 8915 | Traf 2/6 | 205263_at; | F |
| | | Complex | | |
| MALT1 | 10892 | Traf 2/6 | 208309_s_at; 210017_at; 210018_x_at; | EEE |
| | | Complex | | |
| TRAF6 | 7186 | Traf 2/6 | 204413_at; | E |
| | | Complex | | |
| TRAF2 | 7189 | Traf 2/6 | 205558_at; | D |
| | | Complex | | |
| PAR1 | 2149 | PAR | 203989_x_at; | B |
| PAR4 | 5074 | PAR | 204004_at; 204005_s_at; 226223_at; 226231_at; | FFFF |
| CRK | 1398 | SAPK/JNK | 202224_at; 202226_s_at; | EE |
| CRKL | 1399 | SAPK/JNK | 206184_at; 212180_at; | BC |
| SHC | 6464 | SAPK/JNK | 201469_s_at; 214853_s_at; | DA |
| SHC3 | 25759 | SAPK/JNK | 213464_at; | B |
| GRB2 | 2885 | SAPK/JNK | 215075_s_at; 223049_at; | EE |
| SOS | 6654 | SAPK/JNK | 212777_at; 212780_at; 229261_at; 230337_at; | CCDDDE |
| | | | 232883_at; 242018_at; | |
| HPK2 | 9448 | SAPK/JNK | 206571_s_at; 218181_s_at; 222547_at; | DDDDDD |
| | | | 222548_s_at; 238769_at; 244846_at; | |
| TAB1 | 10454 | TAK1 | 203901_at; 233679_at; | DE |
| | | Complex | | |
| TAB2 | 23118 | TAK1 | 210284_s_at; 212184_s_at; 243557_at; | BAA |
| | | Complex | | |
| TAB3 | 257397 | TAK1 | 227357_at; | E |
| | | Complex | | |
| TAK1 | 6885 | TAK1 | 206853_s_at; 206854_s_at; 211536_x_at; | BBBB |
| | | Complex | 211537_x_at; | |
| NLK | 51701 | NFkB | 218318_s_at; 222589_at; 222590_s_at; | DDD |
| IKK1 | 1147 | NFkB | 209666_s_at; | E |
| IKK2 | 3551 | NFkB | 209341_s_at; 209342_s_at; 211027_s_at; | CDD |
| IKK3 | 8517 | NFkB | 209929_s_at; 36004_at; | EC |
| NFKB | 4790 | NFkB | 209239_at; 239876_at; | CD |
| RELA | 5970 | NFkB | 201783_s_at; 209878_s_at; | CC |
| MKK4 | 6416 | JNK | 203265_s_at; 203266_s_at; | BE |
| MKK7 | 5609 | JNK | 216206_x_at; 226053_at; | DD |
| JNK | 5599 | JNK | 226046_at; 226048_at; | EE |
| MAPK9 | 5601 | JNK | 203218_at; 210570_x_at; 225781_at; | EED |
| MAPK10 | 5602 | JNK | 204813_at; | B |
| DUSP8 | 1850 | JNK | 206374_at; | C |
| IRS1 | 3667 | JNK | 204686_at; 235392_at; | DD |
| SMC | 9918 | JNK | 201774_s_at; | C |
| HNRPK | 3190 | JNK | 200097_s_at; 200775_s_at; 200097_s_at; | EEE |
| TP53 | 7157 | JNK | 201746_at; 211300_s_at; | DD |
| ATF2 | 1386 | JNK | 205446_s_at; 212984_at; | DE |
| ELK1 | 2002 | JNK | 203617_x_at; 210376_x_at; 210850_s_at; | CBB |
| cJUN | 3725 | JNK | 201464_x_at; 201465_s_at; 201466_s_at; | ABAB |
| | | | 213281_at; | |
| NFAT4 | 4775 | JNK | 207416_s_at; 210555_s_at; 210556_at; | DDD |
| NFATC1 | 4772 | JNK | 208196_x_at; 210162_s_at; 211105_s_at; | ECC |
| NLK | 51701 | WNT | 218318_s_at; 222589_at; 222590_s_at; | DDD |
| CTBP1 | 1487 | WNT | 203392_s_at; 212863_x_at; 213980_s_at; | CCC |
| CBP | 1387 | WNT | 202160_at; 237239_at; | CD |
| TCF | 3172 | WNT | 208429_x_at; 214832_at; 214851_at; | BBBE |
| | | | 216889_s_at; | |
| GROUCHO | 7091 | WNT | 204872_at; 214688_at; 216997_x_at; | DDDDD |
| | | | 233575_s_at; 235765_at; | |
| | | | 213763_at; 219028_at; 225097_at; | |
| HIPK2 | 28996 | WNT | 225116_at; 225368_at; | BBBBB |
| cMYB | 4602 | WNT | 204798_at; | E |
| ATF2 | 1386 | ALK | 205446_s_at; 212984_at; | DE |
| MAP2K6 | 5608 | p38 | 205698_s_at; | E |
| p38MAPK | 5594 | p38 | 208351_s_at; 212271_at; 224621_at; | BBD |
| MNK1 | 8569 | p38 | 209467_s_at; 243256_at; | CB |
| PRAK | 8550 | p38 | 212871_at; | E |
| HSP27 | 6294 | p38 | 201747_s_at; 201748_s_at; 213635_s_at; | CCB |
| MAPKAP2 | 9261 | p38 | 201460_at; 201461_s_at; 215050_x_at; | BBB |
| STAT1 | 6772 | p38 | 200887_s_at; 209969_s_at; AFFX- | AAAAAAAAA |
| | | | HUMISGF3A/M97935_3_at; | AA |
| | | | AFFX-HUMISGF3A/M97935_5_at; | |
| | | | AFFX-HUMISGF3A/M97935_MA_at; | |
| | | | AFFX-HUMISGF3A/M97935_MB_at; | |
| | | | 232375_at; | |
| | | | AFFX-HUMISGF3A/M97935_3_at; | |
| | | | AFFX-HUMISGF3A/M97935_5_at; | |
| | | | AFFX-HUMISGF3A/M97935_MA_at; | |
| | | | AFFX-HUMISGF3A/M97935_MB_at; | |
| MAX | 4149 | p38 | 208403_x_at; 209331_s_at; 209332_s_at; | DDCDBB |
| | | | 210734_x_at; 214108_at; 222174_at; | |
| MYC | 4609 | p38 | 202431_s_at; | E |
| ELK1 | 2002 | p38 | 203617_x_at; 210376_x_at; 210850_s_at; | CBB |
| CHOP | 2521 | p38 | 200959_at; 217370_x_at; 231108_at; | CCC |
| MEF2 | 4205 | p38 | 208328_s_at; 212535_at; 214684_at; 239571_at; | DDDDD |
| | | | 242176_at; | |
| MEF4 | 4207 | p38 | 205124_at; | F |
| MEF5 | 4208 | p38 | 207968_s_at; 209199_s_at; 209200_at; | DDDDDDD |
| | | | 236395_at; 239938_x_at; 239966_at; 244230_at; | |
| ATF2 | 1386 | p38 | 205446_s_at; 212984_at; | DE |
| MSK1 | 9252 | p38 | 204633_s_at; 204635_at; | EE |
| HMG14 | 3150 | p38 | 200943_at; 200944_s_at; | FF |
| CREB | 1385 | p38 | 204312_x_at; 204313_s_at; 204314_s_at; | EEEC |
| | | | 214513_s_at; | |

In still another aspect, the invention includes a method of selecting a patient having a tumour that is susceptible to treatment with a TAK1 inhibitor. The method can include determining if the patient has a genetic mutation of a t(14;18)(q32;q21) translocation, a t(11;18)(q21;q21) translocation, a t(1;14)(p22;q32) translocation, or amplification of chromosome 18, whereby the presence of a mutation indicates the tumour is susceptible to treatment.

Alternatively, the method can include determining if the patient has a deregulated TAK1 signalling transduction molecule, wherein the presence of the deregulated TAK1 signalling transduction molecule is an indication that the patient is susceptible to treatment with a TAK1 inhibitor.

In any of the methods described herein, the TAK1 inhibitor can be administered either as a single agent or in combination with other anti-cancer agents or anti-cancer antibodies.

In another aspect, the invention includes a kit for predicting a patient's response to a TAK1 inhibitor, said kit comprising (a) one or more phospho-specific antibodies against a TAK1 signal transduction molecule, and (b) a reagent suitable for detecting binding of said antibodies to the TAK1 signal transduction molecule.

In yet another aspect, the invention includes methods to classify a tumor as a TAK1 inhibitor sensitive tumor. By measuring the relative levels of particular deregulated TAK1 signalling transduction genes or proteins in tumour tissue it is possible to determine if the tumor is responsive to a TAK1 inhibitor. The present invention can be used to predict the suitability of administering a TAK1 inhibitor to a cancer patient.

According to one aspect of the present invention there is provided a method of selecting a mammal having or suspected of having a tumour for treatment with a TAK1 inhibitor drug. The method includes providing a biological sample from a subject having a B cell tumour, a solid tumor, or a T cell leukemia and testing the biological sample for expression of any one of the genes listed in Table 1 or Table 2, or their gene products, thereby to predict an increased likelihood of response to the TAK1 inhibitor drug. In one embodiment, the method includes testing the biological sample for at least 5, 10, 20, 30, 40, 50 or 100 of the genes listed in Table 1 or Table 2.

### BRIEF DESCRIPTION OF THE DRAWINGS

FIG. 1 shows a schematic of the TAK1 signaling pathway in B cell lymphocytes (BCR) and T cell lymphocytes (TCR).

FIG. 2 shows a bar chart showing the effect of TAK1 knock down by shRNA on the viability of B-cell lymphoma cells with a t(14;18)(q32;q21) translocation.

FIG. 3 shows a bar chart showing the effect of a TAK1 kinase inhibitor on the viability of B-cell lymphoma cells with a t(14;18)(q32;21) translocation.

### DETAILED DESCRIPTION

The present invention is based, in part, on the finding that certain lymphomas, in particular B cell tumors, solid tumors or T cell leukemia's, are selectively responsive to a TAK1 inhibitor.

Moreover, the invention includes identifying tumours carrying particular mutations including a t(14;18)(q32;q21) translocation, a t(11;18)(q21;q21) translocation, a t(1;14)(p22;q32) translocation, an amplification of chromosome 18, an addition of chromosome 18q21, an amplification of specific regions (detected by comparative genomic hybridization), changes in the subcellular localization, over- or under-expression of a deregulated TAK1 signalling transduction molecule, or posttranscriptional modifications in the proteins containing TAK1 pathway signaling molecules including MALT1, BCL-10, TAK1, TRAF6, CARD 11, IRAK1, TAB 1, TAB2, TRAF2, IRAK4, API1, API2, API3, API4 (survivin), BCL2 or NF-kB target genes or deletion of specific regions containing API2. NF-kB target genes include any gene that is regulated by the NF-kB transcription factor, for example a set of NF-KB target genes is provided in the reference Dave S S et. al. N. Engl. J. Med. 2006; 354(23): 2431-42. These tumours have been identified to be particularly susceptible to treatment using a TAK1 inhibitor.

The identification of the above particular mutations of the present invention can be used to determine if a patient is a responder or non-responder to a TAK1 inhibitor. By responders and non responders it is meant objective tumour responses according to the Union International Contre le Cancer/World Health Organization (U ICC/WHO) criteria are categorised as follows: complete response (CR): no residual tumour in all evaluable lesions; partial response (PR): residual tumour with evidence of chemotherapy-induced 50% or greater decrease under baseline in the sum of all measurable lesions and no new lesions; stable disease (SD): residual tumour not qualified for CR; and progressive disease (PD): residual tumour with evidence of 25% or greater increase under baseline in the sum of all measurable lesions or appearance of new lesions. As defined herein non-responders are PD.

### Deregulated TAK1 Signal Transduction Molecules

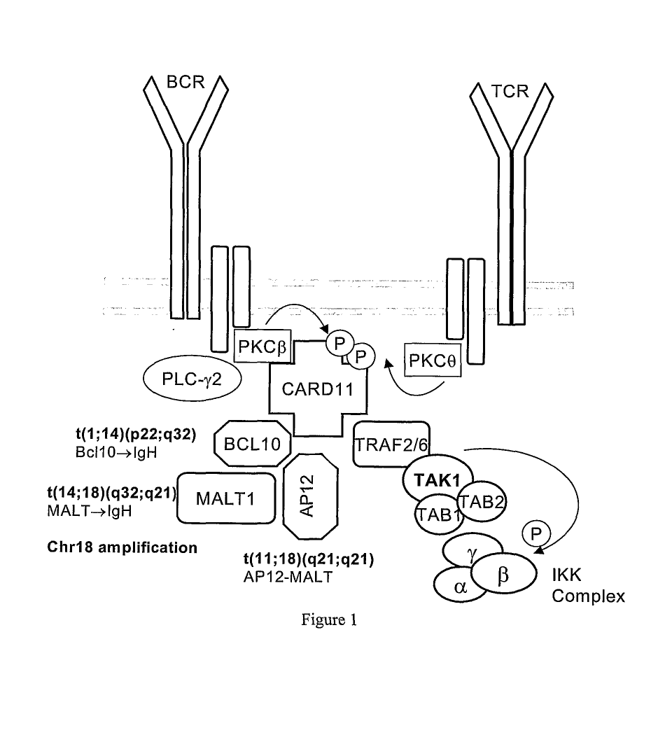

The invention further includes identifying a tumour for a deregulated TAK1 signaling transduction molecule. A deregulated TAK1 signaling transduction molecule is any molecule that is directly or indirectly modified, for example activated or deactivated, in the MALT pathway compared to a normal cell. See FIG. 1. A deregulated TAK1 signaling molecule also includes tumor cells that have a deregulated TAK1 signaling molecule, for example, molecules that are over or under-expressed in signalling pathways involving TAK1. Such signalling pathways include antigen receptor signalling on T and B cells, IL-1 and TLR family signalling, TNF signalling, etc.

Tumours having deregulated TAK1 signaling molecules have been identified to be particularly susceptible to treatment using a TAK1 inhibitor. The present invention includes a number of different biomarkers that can be used to predict a patient's responsiveness to a TAK1 inhibitor. The biomarkers of the invention include genetic mutations whereby the presence of a mutation indicates that the tumour is susceptible to treatment. Examples of genetic mutations that lead to deregulated TAK1 signaling are the t(14;18)(q32;q21) translocation that causes MALT1 (and BCL2) to be overexpressed, the t(11;18)(q21;q21) translocation that results in a fusion protein of MALT1 with API2 and the t(1;14)(p22;q32) translocation that results in overexpression of BCL-10. Further examples of genetic mutations that lead to deregulated TAK1 signaling include amplification of chromosome 18 resulting in overexpression of MALT1 or BCL2, and amplifications or deletions of specific regions identified by comparative genomic hybridization containing key components of the TAK1 signalling pathway including MALT1, BCL-10, TAK1, TRAF6, TRAF2, TAB1, TAB2, CARD11, IRAK1, IRAK4, API1, API2, API3, API4 and NF-kB target genes.

The biomarkers of the invention also include deregulated TAK1 signalling transduction molecules, wherein the presence of the deregulated TAK1 signalling transduction molecule is an indication that the patient is susceptible to treatment with a TAK1 inhibitor. Examples of deregulated TAK1 signal transduction molecules include molecules modified by posttranslational alterations such as phosphorylation including phosphorylated TAK1, phosphorylated IKKbeta, phosphorylated p65, phosphorylated MKK4, phosphorylated MKK6, phosphorylated p38 and phosphorylated JNK. Other molecules that serve as deregulated TAK1 signal transduction molecules include molecules modified by ubiquitinylation including ubiquitinylated TRAF6, ubiquitinylated IKKgamma and ubiquitinylated IkappaBalpha. Other markers that serve as deregulated TAK1 signal transduction molecules include alterations in subcellular localization such as translocation of molecules such as BCL-10, API4, p65 and RelA from the cytoplasm to the nucleus.

A deregulated TAK1 signaling molecule also includes any molecule that is over- or under-expressed in the TAK1 signalling pathway. By measuring the relative levels of one or more deregulated TAK1 signaling molecules as shown in Table 1 or Table 2, i.e., measuring gene expression or protein expression or activity in a tumour tissue it is possible to determine if the tumor is responsive to a TAK1 inhibitor. The present invention can thus be used to predict the suitability of administering a TAK1 inhibitor to a cancer patient.

### Assays

The present invention provides a number of biomarkers that can be used to predict a patient's responsiveness to a TAK1 inhibitor. One exemplary method for detecting the presence of a biomarker includes obtaining a tumour sample from a test subject and determining for the presence of the biomarker.

Any appropriate sample can be used to determine for the presence of a biomarker of the invention. In one example the sample is a suspected B cell tumour and determination of whether that tumour has a genetic mutation is performed. Examples of genetic mutations include a t(14;18)(q32;q21) translocation, a t(11;18)(q21;q21) translocation, a t(1;14)(p22;q32), an amplification of chromosome 18, or addition of chromosome 18q21. These markers can be characterized by fluorescent in situ hybridization, comparative genomic hybridization (CGH) and cDNA microarrays for gene expression profiling and copy number changes.

### Detection of a Deregulated TAK1 Signal Transduction Pathway Molecule

Means of determining if a sample has a genetic mutation are known in the art. These methods include those described or claimed in the following publications, the entire disclosures of which are incorporated by reference herein. Methods to determine if the tumor has an amplification of chromosome 18 are described in Haematologica. 2006; 91(2): 184-91. A t(14;18) translocation can be determined interphase fluorescence in situ hybridization (FISH) as described by Godon A et. al, Leukemia. 2003; 17(1):255-9 or by Farter J L et. al, 1: Diagn Mol Pathol. 2001; 10(4):214-22. A t(14;18)(q32;q21) translocation can be determined as described in Davies et al., Chromosome Res. 2005; 13(3):237-48. The expression of AP12-MALT1 mRNA can be studied using reverse transcriptase (RT)-polymerase chain reaction (PCR) and nested PCR as described in Sanchez-Izquierdo D et. al Blood 2003 101: 11 4539-4546 and Ye H et. al Journal of pathology 2005; 205: 293-301. A t(11;18)(q21;q21) translocation can be detected by RT-PCR of the AP12-MALT1 fusion transcripts and a t(14;18)(q21;q21) translocation can be detected by interphase fluorescence in situ hybridisation (FISH; Vysis Abott Labs).

The procedures and reagents needed to determine for a deregulated TAK1 signal transduction molecule are known in the art. In one example, an agent of interest that can be used to detect a deregulated TAK1 signal transduction molecule includes any molecule such as a peptidomimetic, protein, peptide, nucleic acid, small molecule, an antibody or other drug candidate, that can bind the protein. In one example, antibodies that are commercially available can be used to detect a deregulated TAK1 signal transduction molecule. For example, phosphorylated (phos) TAK1, Phos IkB, Phos IKK, Phos P38 can be measured by using Phospho specific antibodies from Cell Signaling USA. Alternatively, a deregulated TAK1 signal transduction molecule such as MALT and BCL-10 can be performed using immunostaining with mouse monoclonal Antibodies.

Typically, the method includes determining from a tumour sample of a test patient for the presence of a deregulated TAK1 signal transduction pathway molecule. For example, phosphorylated-TAK1 can be visualized by reacting the proteins with antibodies such as monoclonal antibodies directed against the phosphorylated serine, threonine or tyrosine amino acids that are present in the proteins. For example, monoclonal antibodies useful for isolating and identifying phosphotyrosine-containing proteins are described in U.S. Pat. No. 4,543,439.

Typically, antibodies used for visualizing a deregulated TAK1 signal transduction molecule can be labeled by any procedure known in the art, for example, using a reporter molecule. A reporter molecule, as used herein, is a molecule which provides an analytically identifiable signal allowing one of skill in the art to identify when an antibody has bound to a protein that it is directed against. Detection may be either qualitative or quantitative. Commonly used reporter molecules include fluorophores, enzymes, biotin, chemiluminescent molecules, bioluminescent molecules, digoxigenin, avidin, streptavidin or radioisotopes. Commonly used enzymes include horseradish peroxidase, alkaline phosphatase, glucose oxidase and beta-galactosidase, among others. The substrates to be used with these enzymes are generally chosen for the production, upon hydrolysis by the corresponding enzyme, of a detectable color change. For example, p-nitrophenyl phosphate is suitable for use with alkaline phosphatase reporter molecules; for horseradish peroxidase, 1,2-phenylenediamine, 5-aminosalicylic acid or toluidine are commonly used. Incorporation of a reporter molecule onto an antibody can be by any method known to the skilled artisan.

After separation and visualizing the proteins, the amount of each protein species may be assessed by readily available procedures. For example, by using Western blot analysis which includes electrophoretically separating proteins on a polyacrylamide gel, and after detecting the separated proteins, the relative amount of each protein can be quantified by assessing its optical density. Alternatively, other methods such as FACS, immunohistochemistry, immunocytochemistry, fluorescence microscopy, ELISA, etc., can be used either for altered expression of naïve, posttranslationally modified proteins or for monitoring the alterations in the subcellular localization of the proteins.

In the methods of the invention one or more deregulated TAK1 signal transduction pathway molecules can be detected. For example, an assay system can be set up which can detect for the presence of multiple deregulated TAK1 signal transduction pathway molecules.

### Expression Profile

The invention also includes a method for determining an expression profile of an appropriate tumour sample to determine if that tumour is likely to be responsive to TAK1 inhibitor treatment. In one example, the present invention includes determining for the level of expression of the genes in Table 1 or Table 2 in the test tumour sample. The gene profile obtained is compared against controls, i.e., expression patterns, which is indicative that a tumour is responsive to TAK1 treatment. The gene sequences of each of the biomarkers listed in Table 1 or Table 2 can be detected using agents that can be used to specifically detect the gene or other biological molecules relating to it, for example, RNA transcribed from the gene or polypeptides encoded by the gene. Exemplary detection agents are nucleic acid probes, which hybridize to nucleic acids corresponding to the gene, and antibodies.

The biomarkers listed in Table 1 or Table 2 are intended to also include naturally occurring sequences including allelic variants and other family members. The biomarkers of the invention also include sequences that are complementary to those listed sequences resulting from the degeneracy of the code and also sequences that are sufficiently homologous and sequences which hybridize under stringent conditions to the genes listed in Table 1 or Table 2. Conditions for hybridization are known to those skilled in the art and can be found in Current Protocols in Molecular Biology, John Wiley & Sons, N.Y. (1989), 6.3.1-6.3.6. A preferred, non-limiting example of highly stringent hybridization conditions are hybridization in 6× sodium chloride/sodium citrate (SSC) at about 45° C., followed by one or more washes in 0.2×SSC, 0.1% SDS at 50-65° C.

By “sufficiently homologous” it is meant a amino acid or nucleotide sequence of a biomarker which contains a sufficient or minimum number of identical or equivalent (e.g., an amino acid residue which has a similar side chain) amino acid residues or nucleotides to a second amino acid or nucleotide sequence such that the first and second amino acid or nucleotide sequences share common structural domains or motifs and/or a common functional activity. For example, amino acid or nucleotide sequences which share common structural domains have at least about 50% homology, preferably 60% homology, more preferably 70%-80%, and even more preferably 90-95% homology across the amino acid sequences of the domains and contain at least one and preferably two structural domains or motifs, are defined herein as sufficiently homologous. Furthermore, amino acid or nucleotide sequences which share at least 50%, preferably 60%, more preferably 70-80% or 90-95% homology and share a common functional activity are defined herein as sufficiently homologous.

The comparison of sequences and determination of percent homology between two sequences can be accomplished using a mathematical algorithm. A preferred, non-limiting example of a mathematical algorithm utilized for the comparison of sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to TRL nucleic acid molecules of the invention. BLAST protein searches can be performed with the XBLAST program, score=50, wordlength=3 to obtain amino acid sequences homologous to the protein sequences encoded by the genes listed in Table 1 or Table 2. To obtain gapped alignments for comparison purposes, Gapped BLAST can be utilized as described in Altschul et al., (1997) Nucleic Acids Research 25(17):3389-3402. When utilizing BLAST and Gapped BLAST programs, the default parameters of the respective programs (e.g., XBLAST and NBLAST) can be used. See http://www.ncbi.nlm.nih.gov. Another preferred, non-limiting example of a mathematical algorithm utilized for the comparison of sequences is the ALIGN algorithm of Myers and Miller, CABIOS (1989). When utilizing the ALIGN program for comparing amino acid sequences, a PAM120 weight residue table, a gap length penalty of 12, and a gap penalty of 4 can be used.

Methods for Determining Nucleic Acid Sequences that are Differentially Expressed in a Subject with Cancer

The invention provides a list of genes or gene products that can be used to produce an expression profile signature which characteristically predicts TAK1 inhibitor sensitivity of a tumour cell. Any method known in the art can be used to determine whether a tumour cell is responsive to treatment with an TAK1 inhibitor.

In one embodiment, the method comprises determining mRNA and/or protein level of the biomarkers of a mammal, such as by Northern blot analysis, reverse transcription-polymerase chain reaction (RT-PCR), in situ hybridization, immunoprecipitation, Western blot hybridization, or immunohistochemistry. According to the method, cells may be obtained from a subject and the levels of the biomarker's protein or mRNA level are determined and compared to a control.

In one embodiment, the method comprises using a nucleic acid probe to determine whether a mammal is responsive to TAK1 inhibition. The method includes:
providing a nucleic acid probe comprising a nucleotide sequence, for example, at least 10, 15, 25 or 40 nucleotides, and up to all or nearly all of the coding sequence which is complementary to a portion of the coding sequence of a nucleic acid sequence listed in Table 1 or Table 2;
obtaining a tissue sample from a mammal having a cancerous cells;
contacting the nucleic acid probe under stringent conditions with RNA obtained from the sample (e.g., in a Northern blot or in situ hybridization assay); and
comparing the amount of hybridization of the probe with RNA derived from; wherein the amount of hybridization is indicative of the presence of cancerous cells in the first tissue sample.

In another example, the methods of the invention include determining expression profiles with microarrays involves the following steps: (a) obtaining a mRNA sample from a subject and preparing labeled nucleic acids therefrom (the “target nucleic acids” or “targets”); (b) contacting the target nucleic acids with an array under conditions sufficient for the target nucleic acids to bind to the corresponding probes on the array, for example, by hybridization or specific binding; (c) optional removal of unbound targets from the array; (d) detecting the bound targets, and (e) analyzing the results, for example, using computer based analysis methods, to indicate whether the mammal is responsive to TAK1 inhibition treatment

In the method detailed above, the method includes obtaining mRNA from the mammal's tumour sample. RNA may be extracted from tissue or cell samples by a variety of methods, for example, guanidium thiocyanate lysis followed by CsCl centrifugation (Chirgwin, et al., Biochemistry 18:5294-5299, 1979). RNA from single cells may be obtained as described in methods for preparing cDNA libraries from single cells (see, e.g., Dulac, Curr. Top. Dev. Biol. 36:245, 1998; Jena, et al., J. Immunol. Methods 190:199, 1996).

The RNA sample can be further enriched for a particular species. In one embodiment, for example, poly(A)+ RNA may be isolated from an RNA sample. In particular, poly-T oligonucleotides may be immobilized on a solid support to serve as affinity ligands for mRNA. Kits for this purpose are commercially available, for example, the MessageMaker kit (Life Technologies, Grand Island, N.Y.).

In one embodiment, the RNA population may be enriched for sequences of interest, as detailed on Table 1 or Table 2. Enrichment may be accomplished, for example, by primer-specific cDNA synthesis, or multiple rounds of linear amplification based on cDNA synthesis and template-directed in vitro transcription (see, e.g., Wang, et al., Proc. Natl. Acad. Sci. USA 86:9717, 1989; Dulac, et al., supra; Jena, et al., supra).

The target molecules may be labeled to permit detection of hybridization of the target molecules to a microarray. That is, the probe may comprise a member of a signal producing system and thus, is detectable, either directly or through combined action with one or more additional members of a signal producing system. Examples of directly detectable labels include isotopic and fluorescent moieties incorporated, usually by a covalent bond, into a moiety of the probe, such as a nucleotide monomeric unit (e.g., dNMP of the primer), or a photoactive or chemically active derivative of a detectable label which can be bound to a functional moiety of the probe molecule.

In other embodiments, the target nucleic acid may not be labeled. In this case, hybridization may be determined, for example, by plasmon resonance (see, e.g., Thiel, et al., Anal. Chem. 69:4948, 1997).

Microarrays for use according to the invention include one or more probes of genes listed in Table 1 or Table 2.

The method described above results in the production of hybridization patterns of labeled target nucleic acids on the array surface. The resultant hybridization patterns of labeled nucleic acids may be visualized or detected in a variety of ways, with the particular manner of detection selected based on the particular label of the target nucleic acid. Representative detection means include scintillation counting, autoradiography, fluorescence measurement, calorimetric measurement, light emission measurement, light scattering, and the like.

One such method of detection utilizes an array scanner that is commercially available (Affymetrix, Santa Clara, Calif.), for example, the 417™ Arrayer, the 418™ Array Scanner, or the Agilent GeneArray™ Scanner. This scanner is controlled from a system computer with an interface and easy-to-use software tools. The output may be directly imported into or directly read by a variety of software applications. Scanning devices are described in, for example, U.S. Pat. Nos. 5,143,854 and 5,424,186.

### Proteins

Detecting for the presence of a protein product encoded by one or more of the biomarker genes listed in Table 1 or Table 2 can be done by using any appropriate method known in the art. For example, an agent of interest that can be used to detect a particular protein of interest, for example using an antibody. The method for producing polyclonal and/or monoclonal antibodies that specifically bind to polypeptides useful in the present invention is known to those of skill in the art and may be found in, for example, Dymecki, et al., (J. Biol. Chem. 267:4815, 1992); Boersma & Van Leeuwen, (J. Neurosci. Methods 51:317, 1994); Green, et al., (Cell 28:477, 1982); and Arnheiter, et al., (Nature 294:278, 1981).

In one embodiment, an immunoassay can be used to quantitate the levels of proteins in cell samples. The invention is not limited to a particular assay procedure, and therefore, is intended to include both homogeneous and heterogeneous procedures. Exemplary immunoassays that may be conducted according to the invention include fluorescence polarization immunoassay (FPIA), fluorescence immunoassay (FIA), enzyme immunoassay (EIA), nephelometric inhibition immunoassay (NIA), enzyme-linked immunosorbent assay (ELISA), and radioimmunoassay (RIA).

In another example, the presence of the marker protein in a tissue sample can be determined using immunohistochemical staining. For such staining, a multiblock of tissue may be taken from the biopsy or other tissue sample and subjected to proteolytic hydrolysis, employing such agents as protease K or pepsin. In certain embodiments, it may be desirable to isolate a nuclear fraction from the sample cells and detect the level of the marker polypeptide in the nuclear fraction.

In yet another embodiment, the invention contemplates using a panel of antibodies that are generated against the marker polypeptides of this invention. Such a panel of antibodies may be used as a reliable diagnostic probe for determining if a tumour is responsive to treatment with an TAK1 inhibitor.

### Data Analysis

To facilitate the sample analysis operation, the data obtained by the reader from the device may be analyzed using a digital computer. Typically, the computer will be appropriately programmed for receipt and storage of the data from the device, as well as for analysis and reporting of the data gathered, for example, subtraction of the background, deconvolution of multi-color images, flagging or removing artifacts, verifying that controls have performed properly, normalizing the signals, interpreting fluorescence data to determine the amount of hybridized target, normalization of background and single base mismatch hybridizations, and the like.

In one embodiment, a system comprises a search function that allows one to search for specific patterns, for example, patterns relating to differential gene expression, for example, between the expression profile of the test tumour cell and the expression profile of a tumour cell that is responsive to treatment with an TAK1 inhibitor. A system may also allow one to search for patterns of gene expression between more than two samples. Comparison of the expression levels of one or more genes characteristic of responsiveness to an TAK1 inhibitor with reference expression levels, for example, expression levels that are characteristic of susceptibility to an TAK1 inhibitor may be conducted using computer systems.

### Subtyping Diffuse Large B-Cell Lymphoma (DLBL/DLBCL) Patients to Determine if the Patient is Sensitive to a TAK1 Inhibitor

The present invention can be used to subtype DLBCL patients in order to determine if the patients are sensitive or likely insensitive to a TAK1 inhibitor. Specifically, patients can be categorized to determine if the patients fall within 3 distinct subclasses based on their expression pattern of TAK1 genes. A patient sample that falls within Groups 1 and 3 as described below are believed to be TAK1 sensitive, while a patient sample that falls within Group 2 is likely to be TAK1 insensitive. A method for subtyping DLBCL patients is described below. Thus, the invention includes providing a test DLBCL sample and determining whether the sample falls within Groups 1, 2 or 3.
The method includes:
mapping genes of Table 1 to Affymetrix probesets based on the annotations available from Affymetrix (http://www.affymetrix.com/analysis/index.affx);
providing an Affymetrix U133A/B gene chip having gene expression data of 176 newly diagnosed diffuse large B cell lymphoma (DLBCL) patients;
verifying data quality such that 113 samples (Table 3) are kept for further analysis; and
performing sample clustering such that three groups generated, wherein samples that fall within Groups 1 and 3 are TAK1 sensitive, while a sample falling within Group 2 is TAK1 insensitive.
To test whether a DLBCL patient sample falls within Group 1, 2 or 3 the method includes:
providing a test DBLCL patient sample;
providing a data verified U133A/B gene chip;
normalizing the test sample with the 113 samples of Table 3;
clustering the test sample and the 113 samples including the sensitivity determining signature gene set as in Table 2, wherein if the test sample falls within Groups 1 and 3 the sample is TAK1 sensitive, whereas if the test sample falls within Group 2 the sample is TAK1 insensitive.

### Details of how to Perform the Above Method are Provided Below:

### 1. TAK1 Pathways Genes

Genes in TAK1 pathways can be assembled based on the public information. The genes that are involved in the signaling of ALK, FAS, MAP kinase, IL-1 receptor, TGF-beta, TNF receptor, thrombin and protease-activated receptor, Toll-like receptor, WNT, and antigen receptor are included. These genes can be mapped to Affymetrix probesets based on the annotations available from Affymetrix (http://www.affymetrix.com/analysis/index.affx) (Table 1)

### 2. Gene Expression Data

The gene expression data of 176 newly diagnosed diffuse large B cell lymphoma (DLBCL) patients generated with Affymetrix U133A/B gene chip are publicly available by Margaret Shipp's group at Dana Faber Cancer Institute (Molecular profiling of diffuse large B-cell lymphoma identifies robust subtypes including one characterized by host inflammatory response Blood 105(5) 1851-1861). The raw data can be downloaded from http://www.broad.mit.edu/cgi-bin/cancer/datasets.cgi, and further processed and analyzed as described below.

### 3. Data Preprocessing and Analysis

### 3.1 QC:

In order to verify data quality, and generate gene expression results, the raw data (.CEL files) of the DLBCL samples can be loaded into Affymetrix Expression Console 1.0 (Affymetrix Inc.) and analyzed using MAS5 algorithm. The following criteria are used to filter out samples with low quality data: 1) scaling factor <4; 2) rawQ <5; 3) 3′/5′ ratio for both actin and GAPDH <5; 4) percentage of present call >20 for chip A or >10 for chip B. As a result of the QC procedure, 113 samples (Table 3) are kept for further analysis.

### 3.2 Normalization:

Array normalization: The parameters for MAS5 algorithm are set to normalize each array using all probesets on the array, and the trimmed mean value for each array is preset to 100.

Probeset normalization: The expression matrix generated by MAS5 can then be further normalized so that the mean of each probeset is centered to zero.

### 3.3 Sample Clustering:

For unsupervised clustering analysis, the normalized expression matrix is loaded into GeneSpring GX 7.3.1 (Agilent Inc.). A 2-way hierarchical clustering is performed using only probeset identifications from table 2. Spearman correlation was used as the similarity measure in the clustering. The result from clustering reveals three subtypes, Group 1, 2 and 3.

### 4. Test DLBCL Samples

New test patient samples can be profiled using affymetrix U133A/B chips. After the data has been inspected following the same quality control (QC) procedure as described above 3.1, they can be added into the affymetrix U133A/B chip data with 113 samples (Table 3). The new test sample set (113 plus test sample) will be analyzed following the same process as outlined above 3.2-3.3. The new test samples will be clustered into one of the Groups 1-3. If the test sample falls within Group 1 and 3, the patient is likely to be TAK1 sensitive. If the test sample falls within Group 2, the patient is likely to be TAK1 insensitive.

TABLE 3

DLBCL.NEW.206
DLBCL.NEW.210
DLBCL.NEW.211
DLBCL.NEW.215
DLBCL.NEW.219
DLBCL.NEW.230
DLBCL.NEW.232
DLBCL.NEW.239
DLBCL.NEW.240
DLBCL.NEW.242
DLBCL.NEW.244
DLBCL.NEW.246
DLBCL.NEW.250
DLBCL.NEW.251
DLBCL.NEW.254
DLBCL.NEW.259
DLBCL.NEW.261
DLBCL.NEW.262
DLBCL.NEW.267
DLBCL.NEW.268
DLBCL.NEW.269
DLBCL.NEW.270
DLBCL.NEW.271
DLBCL.NEW.272
DLBCL.NEW.277
DLBCL.NEW.279
DLBCL.NEW.280
DLBCL.NEW.282
DLBCL.NEW.283
DLBCL.NEW.284
DLBCL.NEW.285
DLBCL.NEW.286
DLBCL.NEW.290
DLBCL.NEW.291
DLBCL.NEW.295
DLBCL.NEW.300
DLBCL.NEW.301
DLBCL.NEW.303
DLBCL.NEW.304
DLBCL.NEW.307
DLBCL.NEW.309
DLBCL.NEW.311
DLBCL.NEW.312
DLBCL.NEW.313
DLBCL.NEW.332
DLBCL.NEW.333
DLBCL.NEW.336
DLBCL.NEW.338
DLBCL.NEW.339
DLBCL.NEW.340
DLBCL.NEW.344
DLBCL.NEW.345
DLBCL.NEW.347
DLBCL.NEW.348
DLBCL.NEW.349
DLBCL.NEW.350
DLBCL.NEW.353
DLBCL.NEW.357
DLBCL.NEW.359
DLBCL.NEW.361
DLBCL.NEW.404
DLBCL.NEW.405
DLBCL.NEW.408
DLBCL.NEW.411
DLBCL.NEW.412
DLBCL.NEW.416
DLBCL.NEW.417
DLBCL.NEW.418
DLBCL.NEW.421
DLBCL.NEW.422
DLBCL.NEW.423
DLBCL.NEW.426
DLBCL.NEW.427
DLBCL.NEW.430
DLBCL.NEW.432
DLBCL.NEW.433
DLBCL.NEW.435
DLBCL.NEW.436
DLBCL.NEW.441
DLBCL.NEW.443
DLBCL.NEW.445
DLBCL.NEW.448
DLBCL.NEW.449
DLBCL.NEW.451
DLBCL.NEW.452
DLBCL.NEW.454
DLBCL.NEW.455
DLBCL.NEW.458
DLBCL.NEW.460
DLBCL.NEW.461
DLBCL.NEW.462
DLBCL.NEW.463
DLBCL.NEW.467
DLBCL.NEW.470
DLBCL.NEW.474
DLBCL.NEW.475
DLBCL.NEW.476
DLBCL.NEW.477
DLBCL.NEW.479
DLBCL.NEW.481
DLBCL.NEW.482
DLBCL.NEW.485
DLBCL.NEW.496
DLBCL.NEW.497
DLBCL.NEW.498
DLBCL.NEW.502
DLBCL.NEW.503
DLBCL.NEW.504
DLBCL.NEW.507
DLBCL.NEW.509
DLBCL.NEW.514
DLBCL.NEW.609
DLBCL.NEW.617

### TAK1 Inhibitors

TAK1 inhibitors are known in the art, for example, the TAK1 inhibitor can include, for example, a peptide, an antibody, an antisense molecule or a small molecule. TAK1 inhibitors useful in the present invention include but are not limited to, those described or claimed in the following publications the entire disclosures of which are incorporated by reference herein. Examples of small molecule TAK1 inhibitors include zearalenones those disclosed in WO 2002048135, TAK1 short interfering RNA (siRNA) are described in Takaesu et. al J Mol. Biol. 2003; 326(1):105-15 and an inactive mutant of TAK1 is described in Thiefes et. al., J Biol. Chem. 2005; 280(30):27728-41.

The TAK1 inhibitor can be administered either as a single agent or in combination with other anti-cancer agents or anti-cancer antibodies including CHOP or rituximab.

### EXAMPLES

### Example 1

### The Following Example was Performed to Determine Inhibition of Cell growth by a TAK1 inhibitor comprising of shRNA Against TAK1.

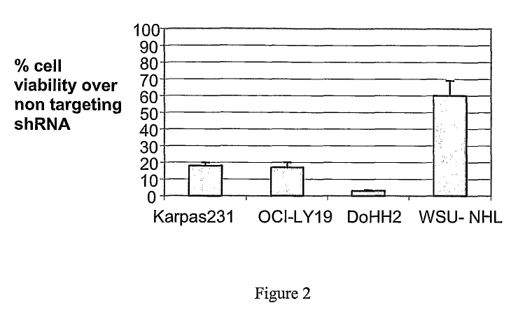

TAK1 shRNAs and scrambled shRNAs were designed using the Ref Seq #: NM_{—}003188 and constructed in to pSIREN RetroQ retroviral vector (Clontech). Initial validation of the shRNAs was done in a HeLa cell line by co-transfection of TAK1 shRNA with NF-KB Luc vector (Clontech's Mercury profiling systems). Takaesu et. al J Mol. Biol. 2003; 326(1):105-15. have demonstrated that TAK1 is critical for the NF-kB activation in HeLa cells. The shRNA construct that showed about 70% inhibition of NF-KB Luc assay and inhibited TAK1 protein levels by 70% was selected for further evaluation of the role of TAK in maintaining the survival of lymphoma cells. This construct along with the scrambled construct was transfected along with gag/pol plasmid and pVSV-G in to the 293T cells. The viral supernatant was harvested and used to infect the lymphoma cells in culture dishes. The four cell lines (OCI-LY19, DOHH2, Karpas231 and WSU-NHL carry the t(14;18) translocation) were plated at 25,000 cells/well in flat-bottomed 24 well plates and treated with 1 ml of viral supernatant from TAK1 shRNA and scrambled shRNA in triplicate and incubated for a total of 72 hours. Following the incubation period, the extent of cell survival was measured by adding 1/10 (vol/vol) AlamarBlue reagent to every well and incubating the plates for a further 4 hours. The reaction was stopped by the addition of SDS to a final concentration of 0.1%. Fluorescence was measured at 545 nm (excitation) and 600 nm (emission). The cell survival data is represented in FIG. 2 as percent of live cells as compared to the scrambled shRNA treated groups.

### Example 2

### The following example was performed to determine inhibition of cell growth by a TAK1 inhibitor comprising a small molecule.

In order to demonstrate that the kinase function of TAK1 is critical for the Lymphoma cell survival, a small molecule inhibitor of TAK1 was tested in the same set of Lymphoma cell lines as above carrying the t(14;18) chromosomal translocation. The chemical name of the compound is 3-[(aminocarbonyl)amino]-5-(4-{[4-(2-methoxyethyl)piperazin-1-yl]methyl}phenyl)thiophene-2-carboxamide.

More specifically, cell lines (OCI-LY19, DOHH2, Karpas231, WSU-NHL and SUDHL4 carry the t(14;18) translocation) were plated at 10,000 cells/well in flat-bottomed 96 well plates and dosed with test compounds in triplicate over a 10 point dosing range from 0 to 30 μmolesL⁻¹. All cell lines were incubated with test compounds for a total of 72 hours. Background levels were determined for a control (undosed) plate within 2 hours of dosing test compounds. Following the dosing period, the extent of proliferation was measured by adding 1/10 (vol/vol) AlamarBlue reagent to every well and incubating the plates for a further 4 hours. The reaction was stopped by the addition of SDS to a final concentration of 0.1%. Fluorescence was measured at 545 nm (excitation) and 600 nm (emission). GI50 values were determined for each test compound across the panel.

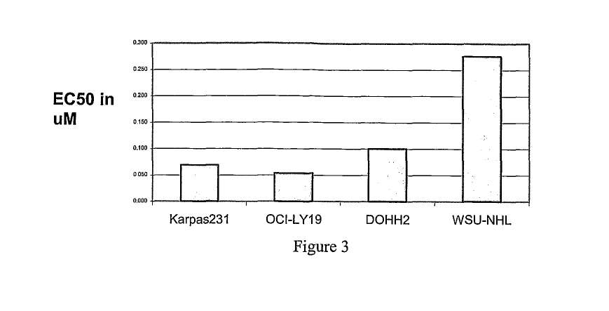

The four cell lines were found to be sensitive to a TAK1 inhibitor. See FIG. 3. The correlation between the sensitivity to TAK1 shRNA and small molecule kinase inhibitor is striking, emphasizing the role of TAK1 kinase activity in the survival of Lymphoma cells carrying the t(14;18).

### Example 3

### The following example was performed to determine inhibition of cellgGrowth by a TAK1 inhibitor comprising a small molecule.

In order to demonstrate that the kinase function of TAK1 is critical for lymphoma cell survival, four small molecule inhibitors, i,e., compound 1 is 2-[(aminocarbonyl)amino]-5-[4-(morpholin-4-ylmethyl)phenyl]thiophene-3-carboxamide, compound 2 is 2-[(aminocarbonyl)amino]-5-[4-(1-piperidin-1-ylethyl)phenyl]thiophene-3-carboxamide, compound 3 is 3-[(aminocarbonyl)amino]-5-[4-(morpholin-4-ylmethyl)phenyl]thiophene-2-carboxamide and compound 4 is 3-[(aminocarbonyl)amino]-5-(4-{[(2-methoxy-2-methylpropyl)amino]methyl}phenyl)thiophene-2-carboxamide, of TAK kinase were tested in a panel of leukaemia and lymphoma cell lines, five of which (OCI-LY19, DOHH2, Karpas231, WSU-NHL and SUDHL4) carry the t(14;18) translocation. The TAK1 inhibitors are known in the art (see for example, WO 2003010158, WO 2003010163 and WO2004063186 the disclosures of which are incorporated by reference herein). All cell lines were plated at 10,000 cells/well in flat-bottomed 96 well plates and dosed with test compounds in triplicate over a 10 point dosing range from 0 to 30 μmolesL⁻¹. All cell lines were incubated with test compounds for a total of 72 hours. Background levels were determined for a control (undosed) plate within 2 hours of dosing test compounds. Following the dosing period, the extent of proliferation was measured by adding 1/10 (vol/vol) AlamarBlue reagent to every well and incubating the plates for a further 4 hours. The reaction was stopped by the addition of SDS to a final concentration of 0.1%. Fluorescence was measured at 545 nm (excitation) and 600 nm (emission). Growth inhibition 50 (GI50) values were determined for each test compound across the panel. See Table 4.

| | TAK1 | | | | | |
|---|---|---|---|---|---|---|
| Compound | pIC50 | OCI-LY19 | DOHH2 | Karpas231 | WSU-NHL | SUDHL4 |
| Number | (enzyme) | DLBCL | FL | B-ALL | B-NHL | DLBCL |
| 1 | 6.9 | 0.124 | 0.127 | 0.30 | 0.37 | 6.39 |
| 2 | 7.3 | 0.005 | 0.014 | 0.14 | 0.25 | 2.42 |
| 3 | 7 | 0.085 | 0.175 | 0.16 | 0.42 | 2.72 |
| 4 | 7.2 | 0.054 | 0.101 | 0.06 | 0.27 | 3.08 |
| | | | | | | ARH77 |
| | | HEL92.1.7 | | | Raji | Plasma |
| Compound | MEC1 | Erythro- | KG1a | Jurkat | Burkitts | cell |
| Number | B-CLL | leukemia | AML | T-ALL | Lymphoma | leukemia |
| 1 | 1.34 | 0.73 | 0.45 | 15.86 | 15.22 | 1.17 |
| 2 | 0.16 | 0.17 | 0.40 | 4.39 | 3.5 | 0.25 |
| 3 | 0.63 | 0.50 | 0.43 | >30 | >30 | 0.48 |
| 4 | 2.99 | 1.47 | 1.09 | 10.09 | >30 | 3.72 |

Table 4 shows GI50 values (μM) for 4 test compounds against a panel of human haematological tumor cell lines.

The TAK1 inhibitor compounds were significantly more potent compared to the mean in four out of five cell lines that carried the t(14;18) chromosomal translocation. This profile was differentiated from other compounds that inhibit other pathways (data not shown). Table 5 shows the GI50 values (μM) for a TAK1 kinase inhibitor against a panel of multiple myeloma tumour cell lines. The results indicate that a distinct set of myeloma cells are responsive to TAK1 inhibitors.

| | | JJN-3 | L-363 | | RPMI-8226 | MOLP-8 | | ARH-77 | KARPAS-620 |
|---|---|---|---|---|---|---|---|---|---|
| Compd | TAK1 | plasma cell | plasma cell | AM0-1 | Multiple | Multiple | IM-9 | plasma cell | plasma cell |
| Number | pIC50 | leukemia | leukemia | plasmacytoma | myeloma | myeloma | B lymph | leukemia | leukemia |
| 4 | 7.2 | 0.52 | 0.35 | 0.91 | 1.60 | 0.09 | 0.21 | 3.72 | 0.14 |

Table 5 shows GI50 values (μM) for compound 4 against a panel of human multiple myeloma cell lines

Table 6 shows the GI50 values (μM) for a TAK1 kinase inhibitor against a panel of human B-cell lymphoma cell lines. The experiments to generate the results for both Tables 5 and 6 were performed as described above. The results indicate that a distinct set of human B-cell tumor cells are responsive to TAK1 inhibitors.

| | | | | NAMALWA | |
|---|---|---|---|---|---|
| Compd | TAK1 | SC-1 | JEKO-1 | Burkitts | MEC-1 |
| Number | pIC50 | FL | MCL | Lymphoma | B-CLL |
| 4 | 7.2 | 2.88 | 0.29 | 2.96 | 2.99 |

| | | | WSU | U937 | Ramos | Raji |
|---|---|---|---|---|---|---|
| | | DERL 2 | DLCL2 | Histiocytic | Burkitts | Burkitts |
| Compd | JVM-3 | T cell | B cell | lymph- | Lymph- | Lymph- |
| Number | B-PLL | lymphoma | lymphoma | oma | oma | oma |
| 4 | 0.14 | 0.31 | 1.95 | 0.12 | 0.21 | >30 |

Table 6 shows GI50 values (μM) for compound 4 against a panel of human B cell lymphoma cell lines

Some of the TAK inhibitors used in the study belong to a large class of thiophene carboxamide ureas that are known to inhibit other enzymes with similar potency against TAK1, such as FLT3, CHK1, ARK5 and Aurora B kinase. Hence in order to rule out any off target effects of TAK1 inhibitors in lymphoma and myeloma cell lines, we further utilized a commercially available TAK1 specific inhibitor, LL-Z-1640-2, which is a (3S,5Z,8S,9S,11E)-8,9,16-trihydroxy-14-methoxy-3-methyl-3,4,9,10-tetrahydro-1H-2-benzoxacyclotetradecine-1,7(8H)-dione (Iris Biotech, GmbH; see WO-00248135). Table 7 shows the GI50 values (μM) for the TAK1 kinase inhibitor, LL-Z-1640-2 against a panel of B-cell lymphoma cell lines.

| | | | | | WSU- | | | | |
|---|---|---|---|---|---|---|---|---|---|
| | | | | OCI- | DLCL | | MEC1 | JVM-3 | |
| | TAK1 | SUDHL4 | KARPAS231 | LY19 | B cell | DOHH2 | B- | B- | JEKO-1 |
| Compd Number | pIC50 | DLBCL | B-ALL | DLBCL | lymphoma | FL | CLL | PLL | MCL |
| LL-Z-1640-2 | 6.8 | 1.64 | 0.95 | 0.23 | 0.02 | 0.39 | 0.73 | 0.71 | 0.07 |

Table 7 shows GI50 values (μM) for another TAK1 kinase inhibitor against a panel of human B cell lymphoma cell lines

Table 8 shows the GI50 values (μM) for the TAK1 kinase inhibitor, LL-Z-1640-2 against a panel of multiple myeloma tumour cell lines. The experiments were performed as described above. The results indicate that, similar to the thiophene carboxamide ureas a distinct set of B-cell lymphoma and myeloma cells are responsive to TAK1 inhibitors.

| | | JJN-3 | L-363 | |
|---|---|---|---|---|
| | TAK1 | plasma cell | plasma cell | AM0-1 |
| Compd | pIC50 | leukemia | leukemia | plasmacytoma |
| LL-Z-1640-2 | 6.8 | 32.43 | 32.43 | 0.57 |

| | | | | ARH-77 | KARPAS- |
|---|---|---|---|---|---|
| | RPMI- | MOLP-8 | | plasma | 620 |
| | 8226 | Multiple | IM-9 B | cell | plasma cell |
| Compd | M.M | myeloma | lymphoblastoid | leukemia | leukemia |
| LL-Z- | 1.41 | 10.44 | 0.02 | 1.24 | ND |
| 1640-2 | | | | | |

Table 8 shows GI50 values (μM) for another TAK1 kinase inhibitor against a panel of human multiple myeloma cell lines

### Example 4

### Genomic Analysis of TAK1 Pathways in DLBCL

### 1. Tak1 Pathways Genes

Genes in Tak1 pathways were assembled based on the public information. The genes that are involved in the signaling of ALK, FAS, MAP kinase, IL1 receptor, TGF-beta, TNF receptor, thrombin and protease-activated receptor, Toll-like receptor, WNT, and antigen receptor were included. The genes were mapped to Affymetrix probesets based on the annotations available from Affymetrix (http://www.affymetrix.com/analysis/index.affx) (Table 1)

### 2. Gene Expression Data

The gene expression data of 176 newly diagnosed diffuse large B cell lymphoma (DLBCL) patients were generated with Affymetrix U133A/B gene chip and were made publicly available by Margaret Shipp's group at Dana Faber Cancer Institute ( ). The raw data were downloaded from http://www.broad.mit.edu/cgi-bin/cancer/datasets.cgi, and further processed and analyzed as described below.

### 3. Data Preprocessing and Analysis

### 3.1 QC:

In order to verify data quality, and generate gene expression results, the raw data (.CEL files) of the DLBCL samples were loaded into Affymetrix Expression Console 1.0 (Affymetrix Inc.) and analyzed using MAS5 algorithm. The following criteria were used the filter out samples with low quality data: 1) scaling factor <4; 2) rawQ <5; 3) 3′/5′ ratio for both actin and GAPDH <5; 4) percentage of present call >20 for chip A or >10 for chip B. As a result of the QC procedure, 113 samples (Table 3) were kept for further analysis.

### 3.2 Normalization:

Array normalization: The parameters for MAS5 algorithm were set to normalize each array using all probesets on the array, and the trimmed mean value for each array was preset to 100. Probeset normalization: The expression matrix generated by MAS5 were further normalized so that the mean of each probeset was centered to zero.

### 3.3 Sample Clustering:

For unsupervised clustering analysis, the normalized expression matrix was loaded into GeneSpring GX 7.3.1 (Agilent Inc.). A 2-way hierarchical clustering was performed using only probeset IDs from table 2. Spearman correlation was used as the similarity measure in the clustering.

### Results:

The 113 newly diagnosed DLBCL samples were separated into 3 distinct subclasses based on their expression pattern of Tak1 genes. The informative genes (the genes that are differentially expressed among the 3 patient subclass) were further divided into 7 groups (A-F) based on their distinct expression patterns. Most of the informative genes in Group 2 are down-regulated compared to the other 2 groups, suggesting the samples in this group represent a patient population that is insensitive to Tak1-targeted therapy.

What is claimed is:

1. A method of inhibiting B cell tumour cell proliferation by contacting a B cell tumour cell with a TAK1 inhibitor.

2. The method of claim 1 wherein the B cell tumour is a non-Hodgkin's lymphoma, a Hodgkin's lymphoma, a chronic lymphocytic leukaemia, or a multiple myeloma.

3. A method of treating a patient having a B cell tumour by administering a TAK1 inhibitor.

4. The method of claim 3 wherein the B-cell tumour is a non-Hodgkin's lymphoma, a Hodgkin's lymphoma, a chronic lymphocytic leukaemia (CLL) or a multiple myeloma.

5. The method of claims 2 or 4 wherein the non-Hodgkin's lymphoma is a follicular lymphoma, a diffuse large B cell lymphoma (DLBCL) of activated B cell (ABC) type, a diffuse large B cell lymphoma (DLBCL) of germinal center B cell (GCB) type, a mantle zone lymphoma (MZL), Mantle cell lymphoma (MCL), Primary mediastinal B-cell lymphoma (PMBCL) or MALT Lymphoma.

6. The method of claim 5 wherein the non-Hodgkin's lymphoma has a t(14;18)(q32;q21) translocation, a t(11;18)(q21;q21) translocation, a t(1;14)(p22;q32), an amplification of chromosome 18, an amplification of chromosome 6, or an amplification, as defined by comparative genomic hybridization, of specific regions of BCL-10, CARD11, TRAF6 or TAK1.

7. The method of claims 2 or 4 wherein the B-cell tumour is CLL.

8. A method of treating a patient having a deregulated TAK1 signalling transduction molecule by administering a TAK1 inhibitor.

9. The method of claim 8 wherein the TAK1 signalling transduction molecule is Malt1, BCL-10, BCL2, TAB1, TAB2, TAK1, TRAF2, TRAF6, TAK1, CARD11, IRAK1, IRAK4, API1, API2, API3, API4 or NFkappaB target genes.

10. A method of inhibiting the growth of a solid tumour by contacting the tumour with a TAK1 inhibitor.

11. The method of claim 10, wherein the solid tumour is selected from the group consisting of a tumour of the head and neck, breast, ovary, lung, pancreas, colon, prostate, or skin.

12. A method of treating a patient having a solid tumour by administering a TAK1 inhibitor.

13. The method of claims 10 or 12, wherein the solid tumour can be a tumour of the head and neck, breast, ovary, lung, pancreas, colon, prostate, liver, or skin.

14. A method of selecting a patient having a tumour that is susceptible to treatment with a TAK1 inhibitor, comprising determining if the patient has a genetic mutation of a t(14;18)(q32;q21) translocation, a t(11;18)(q21;q21) translocation, a t(1;14)(p22;q32) translocation, or an amplification of chromosome 18, whereby the presence of a mutation indicates the tumour is susceptible to treatment.

15. A method of selecting a patient having a tumour that is susceptible to treatment with a TAK1 inhibitor, comprising determining if the patient has a deregulated TAK1 signalling transduction molecule, wherein the presence of the deregulated TAK1 signalling transduction molecule is an indication that the patient is susceptible to treatment with a TAK1 inhibitor.

16. A method of inhibiting proliferation of a T cell leukemia and T-cell lymphomas by contacting a T cell leukaemia and T-cell lymphoma with a TAK1 inhibitor.

17. The method of claim 16, wherein the T cell leukemia is a T-cell acute lymphoblastic leukemia (T-ALL), or T-cell lymphomas, for example, peripheral T-cell lymphoma (PTCL), T-cell lymphoblastic lymphoma (T-CLL), cutaneous T-cell lymphoma (CTCL) and adult T-cell lymphoma (ATCL).

18. A method of selecting a mammal having or suspected of having a tumour for treatment with a TAK1 inhibitor drug, the method comprising providing a biological sample from a subject having cancer and testing the biological sample for expression of any one of the genes listed in Table 1, or their gene products, thereby to predict an increased likelihood of response to the TAK1 inhibitor drug.
